(19) United States
(12) Patent Application Publication
(10) Pub. No.: US 2024/0407373 A1
(43) Pub. Date: Dec. 12, 2024

(54) PHAGE FOR LYSING BURKHOLDERIA GLADIOLI AND USE THEREOF

(71) Applicant: Hefei University of Technology, Hefei (CN)

(72) Inventors: Yingwang YE, Hefei (CN); Ting WANG, Hefei (CN); Na LING, Hefei (CN); Bin CHENG, Hefei (CN); Rui JIAO, Hefei (CN); Xiyan ZHANG, Hefei (CN); Danfeng ZHANG, Hefei (CN)

(73) Assignee: Hefei University of Technology, Hefei (CN)

(21) Appl. No.: 18/378,412

(22) Filed: Oct. 10, 2023

(30) Foreign Application Priority Data
Jun. 8, 2023 (CN) 2023106808478

(51) Int. Cl. A01N 63/40; C12N 7/00

(52) U.S. Cl. CPC A01N 63/40; C12N 7/00; C12N 2795/00031

(57) ABSTRACT

The present disclosure provides a phage for lysing Burkholderia gladioli and use thereof, and belongs to the technical field of phages. The phage for lysing Burkholderia gladioli provided by the present disclosure is Burkholderia gladioli phage vB_BglM_WTB with an accession number of CCTCC M 2023525, which is a novel phage. The phage provided by the present disclosure has a strong lytic effect on the Burkholderia gladioli with higher temperature tolerance and wider acid-base tolerance range and effectively kills the Burkholderia gladioli on food surface, providing a new strategy for controlling the Burkholderia gladioli in food processing and environment.

### CROSS REFERENCE TO RELATED APPLICATION

This patent application claims the benefit and priority of Chinese Patent Application No. 202310680847.8, filed with the China National Intellectual Property Administration on Jun. 8, 2023, the disclosure of which is incorporated by reference herein in its entirety as part of the present application.

### TECHNICAL FIELD

The present disclosure belongs to the technical field of phages, and particularly relates to a phage for lysing Burkholderia gladioli and use thereof.

### BACKGROUND

Burkholderia gladioli is a non-spore-forming Gram-negative brevibacterium. Its optimum growth temperature is 37° C. and its optimum pH is 5-6. Burkholderia gladioli is widely distributed in nature. Natural environment, like soil and plants, and animals may possibly be sources of contamination of Burkholderia gladioli. The food may be contaminated, and rice, onion, and the like may be contaminated by this bacterium. Although a plurality of Burkholderia gladioli strains are nontoxic in terms of environmental protection, some species may cause diseases in animals and plants. Recent studies have found that this bacterium has four pathogenic variants, of which Burkholderia gladioli pathovar cocovenenans produces toxic bongkrekic acid. This pathogenic variant is present in fermented cereal-based products, and the other three ones are pathogenic to plants.

Burkholderia gladioli pathovar cocovenenans is a highly lethal foodborne pathogen, and the latent period thereof is usually 30 min to 12 h, and even lasts for 1-2 days in rare cases. The poisoning of this bacterial species has prominent regional characteristics, which was mostly reported in Guangxi, mountainous areas of Yunnan and Guizhou, and northeastern China. This is related to consumption of snow fungus (Tremella fuciformis), black fungus (Auricularia auricula), diaojiangba, and fermentive corn flour by local residents. In October 2020, nine residents from Jixi, Heilongjiang Province died from poisoning due to eating Burkholderia gladioli-contaminated suantangzi, a local special food, arousing high social concern. Bongkrekic acid produced by this bacterium is a leading cause of serious poisoning and death, and the optimum temperature that produces this toxin is 26-28° C. Bongkrekic acid is a small molecular fatty acid with extremely strong heat resistance. Boiling at 100° C. and or autoclaving still cannot destroy its toxicity. Bongkrekic acid can lead to poisoning after intake, and cause serious damage to human liver, kidneys, heart, brain, and other important organs.

The rate of poisoning caused by Burkholderia gladioli is approximately at least 50%, with a mortality of at least 80%, but there is no related literature report of drugs for prophylaxis and treatment of this infection so far. Therefore, control of this bacterium and toxin thereof is a problem to be resolved urgently in food industry.

Phage is a special virus that can specifically infect bacteria, fungi and actinomycetes. With strong specificity, phages only aim at specific pathogens and will not disrupt a normal microecological balance. Once a phage infects a host, the host is lysed to release substantial phage progenies, which have an exponential growth potential in the host and achieve a bactericidal effect in a short time. Compared with physical and chemical control, phages have advantages of fast acting, strong effect and slight side effects when used in controlling bacteria in food. Based on the above advantages, phages have broad application prospect in bacterial control.

### SUMMARY

In view of this, an objective of the present disclosure is to provide a phage strain for specifically lysing Burkholderia gladioli. The phage strain is capable of effectively inhibiting the Burkholderia gladioli, has no lytic effect on non-Burkholderia gladioli strains, does not contain a virulence gene and a drug resistance gene, and is safe and reliable.

Another objective of the present disclosure is to provide a lysate and a preparation containing a Burkholderia gladioli phage and use thereof in controlling Burkholderia gladioli.

To achieve the above objective, the present disclosure provides the following technical solutions:

The present disclosure provides a phage. The phage is Burkholderia gladioli phage vB_BglM_WTB with an accession number of CCTCC NO: M 2023525.

In the present disclosure, a genome of the phage has a full length of 68,541 bp and contains no virulence gene or drug resistance gene.

In the present disclosure, the phage is stably active at pH 3-11 and has a tolerable temperature of 25-65° C.

The present disclosure further provides a lysate, and the lysate is a lysate of the foregoing phage.

The present disclosure further provides a preparation, and the preparation includes the foregoing phage or the foregoing lysate.

The present disclosure further provides use of the foregoing phage, the foregoing lysate or the foregoing preparation in controlling Burkholderia gladioli.

Preferably, the use is intended to control the Burkholderia gladioli in food.

Preferably, the food is one selected from the group consisting of black fungus (Auricularia auricula), snow fungus (Tremella fuciformis), shiitake mushroom (Lentinula edodes), and golden needle mushroom (Flammulina velutipes).

Preferably, the phage has a multiplicity of infection (MOI) of 1:10,000-1:100.

Preferably, the phage, the lysate or the preparation is in contact with the Burkholderia gladioli.

Preferably, a pH value of a contact medium is 3-11, and contact time is at least 2 h.

The present disclosure has the following beneficial effects:

In the present disclosure, Burkholderia gladioli phage vB_BglM_WTB is isolated from pathogenic Burkholderia gladioli ATCC 33664 as a host, and the phage has a strong lytic effect on pathogenic Burkholderia gladioli in food like black fungus. In the present disclosure, the Burkholderia gladioli phage vB_BglM_WTB can specifically lyse the Burkholderia gladioli, and has no lytic effect on non-Burkholderia gladioli strains. Comparative genomic analysis and phylogenetic analysis have found that this phage has the most similarity to Burkholderia phage Maja, and the similarity is 25.7% (<50%). Therefore, it is recommended that the phage constitutes a new genus.

In the present disclosure, the isolated Burkholderia gladioli phage vB_BglM_WTB is a virulent phage isolated from nature. This phage is free of a virulence gene and a drug resistance gene, safe and reliable, and has excellent temperature tolerance and pH stability. The present disclosure does not make any genetic modification to this phage.

The Burkholderia gladioli phage vB_BglM_WTB provided by the present disclosure can effectively kill Burkholderia gladioli on the surface of food like black fungus. The Burkholderia gladioli phage vB_BglM_WTB has a strong lytic effect on the Burkholderia gladioli in food like black fungus. At 4° C., sterilization rate at 6 h reaches 99.94%, and after treatment for 12 h, the sterilization rate can still reach 99.80%; at 25° C., sterilization rate at 2 h reaches 99.90%, and after treatment for 12 h, the sterilization rate can still reach 99.97%. Therefore, the Burkholderia gladioli phage vB_BglM_WTB can serve as an effective bactericide in food like black fungus. An action mode of the present disclosure may be soaking in a liquid, which is particularly suitable for food that needs to be cleaned, for example, black fungus, snow fungus, shiitake mushroom, and golden needle mushroom.

### Deposit of Biological Material:

Burkholderia gladioli phage vB_BglM_WTB provided by the present disclosure is deposited at China Center for Type Culture Collection (CCTCC), Wuhan University, No. 299 Bayi Road, Wuchang District, Wuhan City, Hubei Province, China on Apr. 12, 2023 with an accession number of CCTCC NO: M 2023525.

### BRIEF DESCRIPTION OF THE DRAWINGS

FIG. 1 is an electron micrograph of Burkholderia gladioli phage vB_BglM_WTB;

FIG. 2 is a picture of Burkholderia gladioli phage vB_BglM_WTB lysing host Burkholderia gladioli ATCC 33664:

FIG. 3 is a heatmap of genes of Burkholderia gladioli phage vB_BglM_WTB;

FIG. 4 is a visual analysis chart of genomes of Burkholderia gladioli phage vB_BglM_WTB, Burkholderia phage BcepF1, and Burkholderia phage Maja;

FIG. 5 shows a phylogenetic tree of major capsid proteins of Burkholderia gladioli phage vB_BglM_WTB;

FIG. 6 shows a one-step growth curve of Burkholderia gladioli phage vB_BglM_WTB;

FIG. 7 is a schematic diagram of pH stability of Burkholderia gladioli phage vB_BglM_WTB;

FIG. 8 is a schematic diagram of thermal stability of Burkholderia gladioli phage vB_BglM_WTB;

FIGS. 9A-B show inhibitory effects of Burkholderia gladioli phage vB_BglM_WTB on Burkholderia gladioli in black fungus at low temperature (4° C.) and room temperature (25° C.), where FIG. 9A shows an antibacterial effect of the Burkholderia gladioli phage vB_BglM_WTB at 4° C., and FIG. 9B shows an antibacterial effect of the Burkholderia gladioli phage vB_BglM_WTB at 25° C.

### DETAILED DESCRIPTION OF THE EMBODIMENTS

The present disclosure provides a phage for lysing Burkholderia gladioli phage vB_BglM_WTB. The Burkholderia gladioli phage vB_BglM_WTB is deposited with an accession number of CCTCC NO: M 2023525.

In the present disclosure, the Burkholderia gladioli phage vB_BglM_WTB is isolated from a sewage sample collected from an intercepting well sewage treatment plant, Huaihe Road, Hefei, and deposited at CCTCC on Apr. 12, 2023. After identification, the Burkholderia gladioli phage vB_BglM_WTB provided by the present disclosure has a regular icosahedral head with a head diameter of approximately 69(±2) nm and a tail length of approximately 108(±2) nm, and belongs to the class Caudoviricetes, with a titer of at least 10⁹ pfu/mL; a genome of the Burkholderia gladioli phage vB_BglM_WTB provided by the present disclosure has a full length of 68,541 bp and a GC content of 60.04% and contains no virulence gene or drug resistance gene. This phage has the most similarity to Burkholderia phage Maja, and the similarity is 25.7% (<50%). Therefore, it is recommended that the phage constitutes a new genus.

In the present disclosure, the Burkholderia gladioli phage vB_BglM_WTB can specifically lyse Burkholderia gladioli, and has no lytic effect on non-Burkholderia gladioli strains. In the present disclosure, the Burkholderia gladioli phage vB_BglM_WTB preferably has an MOI of 1:10,000-1:100 and most preferably 1:10,000, and possesses excellent temperature tolerance and pH stability. The titer is stable at pH 3-11 and a temperature of 25-65° C.

The present disclosure further provides a lysate of the foregoing phage. As an implementation, the foregoing phage is cultured in a culture medium supplemented with a host, and the host is removed to obtain a phage lysate. The host may be optionally removed by filtration or centrifugation. The host is Burkholderia gladioli, and the culture medium is optionally Tryptic Soy Broth (TSB).

The present disclosure further provides a preparation, and the preparation includes the foregoing phage or the foregoing lysate.

The preparation provided by the present disclosure further includes other active antibacterial ingredients for inhibiting or killing Burkholderia gladioli, including but not limited to microbes, Chinese herb extracts or compositions, natural compounds, and chemically synthesized compounds or compositions thereof.

A dosage form of the preparation provided by the present disclosure is not particularly limited, and liquid, solid, semisolid or gaseous preparation may be used. According to different dosage forms, the preparation provided by the present disclosure further includes preparation acceptable excipients. The present disclosure has no particular limitation on excipients, including but not limited to one or more selected from the group consisting of carriers, diluents, vehicles, preservatives, surfactants, and antioxidants.

The present disclosure further provides use of the foregoing phage, the foregoing lysate or the foregoing preparation in controlling Burkholderia gladioli.

In the present disclosure, the controlling includes prevention and management. The phage, the lysate or the preparation provided by the present disclosure is in contact with the Burkholderia gladioli for controlling, and may be used in various forms, including but not limited to soaking, applying, spraying and other forms of applying on a surface of an object. The surface may be one selected from the group consisting of contaminated sites and expected contaminated sites.

The phage, the lysate or the preparation provided by the present disclosure may be used for controlling the Burkholderia gladioli in food. The present disclosure has no particular limitation on food type, and soakable fungal food like black fungus, snow fungus, shiitake mushroom, and golden needle mushroom may be used.

When the phage, the lysate or the preparation provided by the present disclosure is used for controlling the Burkholderia gladioli, the phage, the lysate or the preparation may be used under refrigeration or at room temperature, and preferably, a refrigeration temperature of 3-8° C. and a room temperature higher than the refrigeration temperature may be used. Preferably, the room temperature is 18-38° C., and further preferably 23-28° C. In the present disclosure, treatment for 2 h can achieve effective bactericidal effect, and the bactericidal effect can last until 12 h after treatment. In the present disclosure, in a preferred example, at 4° C., sterilization rate at 6 h reaches 99.94%, and after treatment for 12 h. the sterilization rate can still reach 99.80%; at 25° C., sterilization rate at 2 h reaches 99.90%, and after treatment for 12 h, the sterilization rate can still reach 99.97%.

When the phage, the lysate or the preparation provided by the present disclosure contacts and controls the Burkholderia gladioli, preferably, a pH value of a contact medium is 3-11. In the foregoing pH range, the phage can maintain high activity against the Burkholderia gladioli. Because the phage has a tolerable temperature of 25-65° C., the phage, the lysate or the preparation provided by the present disclosure shall be stored or used at a temperature lower than 65° C.

The technical solutions provided by the present disclosure will be described in detail below with reference to examples, but they should not be construed as limiting the claimed scope of the present disclosure.

In the following examples, unless otherwise specified, all methods are conventional.

All materials and reagents used in the following examples may be commercially available, unless otherwise specified.

### Example 1

Isolation and Identification of Burkholderia gladioli Phage

### (1) Phage Isolation and Identification

In the present disclosure, sewage samples used in the experiment were collected from an intercepting well sewage treatment plant, Huaihe Road, Hefei in 2022.

Sample treatment: A sewage sample was dispensed into a 50 mL centrifuge tube and centrifuged at 10,000 rpm for 10 min to remove larger solid particles and some bacteria; bacteria were removed by filtering through a 0.22 μm millipore filter, the treated sewage sample was transferred into a clean and aseptic container, added with a volume of magnesium sulfate, stirred and mixed well, and left to stand for 15-20 min; the magnesium sulfate mixture was vacuum filtrated to discard the supernatant; the millipore filter was collected to cut into pieces and put in a clean and sterile beaker, added with a volume of eluent, sonicated for 5 min, and centrifuged in a centrifuge tube at 4,000 g for 2 min to collect a supernatant; the supernatant was filtered through a 0.45 μm millipore filter and transferred into a new aseptic container, and this filtrate is a phage stock solution; 500 μL of phage stock solution, 500 μL of TSB and 50 μL of logarithmic Burkholderia gladioli suspension were mixed well and shake-cultured at 37° C. for 8 h. The culture medium was centrifuged at 4,000 g for 15-20 min and filtered through a 0.45 μm millipore filter into an aseptic centrifuge tube, and a phage lysate was obtained. Subsequently, with Burkholderia gladioli ATCC 33664 as a host, the plaque assay was used to identify whether there was a phage. In case of a plaque, a phage is present; otherwise, it is indicated that no phage is isolated and re-screening is needed.

Phage purification: The size and shape of the initially isolated plaque were inconsistent, and further purification was needed. A single, uniformly shaped, clear and transparent plaque was picked from a plaque-containing double-layer agar plate, placed in 1 mL of SM buffer at 4° C. overnight, and centrifuged at 4,000 g for 15-20 min; a supernatant was collected and filtered through a 0.45 μm millipore filter; the filtrate was appropriately diluted and spread on a double-layer agar plate with a logarithmic Burkholderia gladioli ATCC 33664 suspension, and the step was repeated 3-4 times; once all plaques on the double-layer agar plate had consistent size, shape and definition, purified phages were obtained.

### (2) Phage Titer Assay

Purified phage lysate was appropriately diluted, 100 μL of each of phage diluents with latter three dilutions and 100 μL of logarithmic host bacterial suspension were pipetted to determine the phage titer using the double-layer agar plate method. Phage titer (pfu/mL)=Number of plaques×Dilution multiple÷0.1.

Results showed that the size, shape and definition of the plaque were consistent, with typical characteristics of lytic phages. The titer of the phage could reach at least 10⁹ pfu/mL.

### (3) Phage Preservation

The phage proliferation liquid was mixed well with 20% (final concentration) polyethylene glycol 8000 (PEG 8000) and 0.5 M (final concentration) sodium chloride solution in a ratio of 1:1 to concentrate the phage at 4° C. overnight; the concentrated solution was centrifuged at 12,000 g for 15-20 min, the supernatant was discarded, and the pellets were dissolved in SM buffer and mixed well with 60% glycerol in a ratio of 1:1, followed by storage at −20° C.

### Example 2

### Electron Microscopic Observation of the Phage

100 μL of phage lysate (titer 10⁹ pfu/mL) was dropped on the membraniferous side of a copper mesh, and the liquid was blotted up with filter paper after 3-5 min; a drop of 2% phosphotungstic acid (PTA) aqueous solution was dropped on the copper mesh for staining for 2-3 min, and the staining solution was blotted up with filter paper; the copper mesh was observed under an electron microscope, and a clear phage image was selected to photograph.

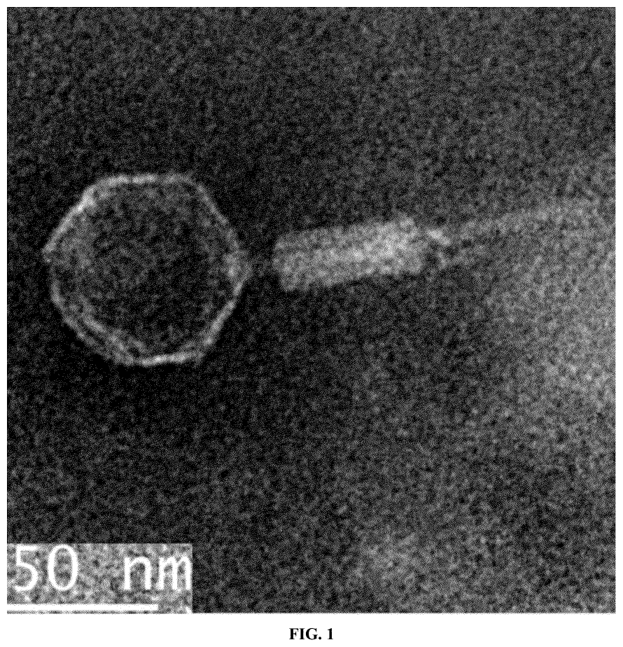

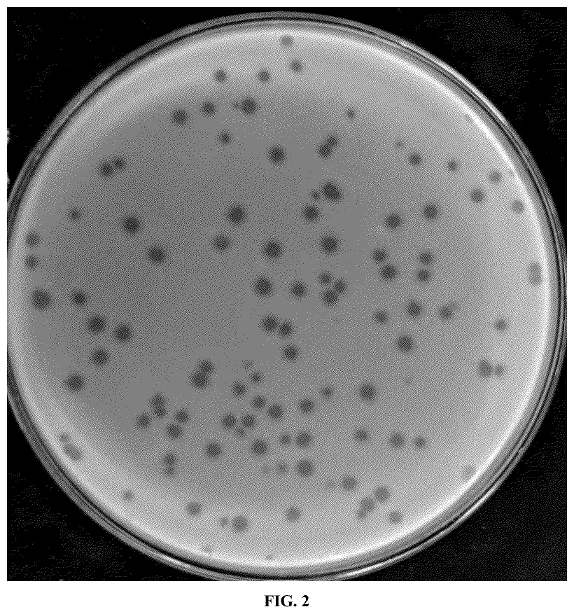

The result is shown in FIG. 1. The phage has a regular icosahedral head with a head diameter of approximately 69(±2) nm and a tail length of approximately 108(±2) nm, and belongs to the class Caudoviricetes.

### Example 3

### Host Spectrum Assay of the Phage

Tryptic Soy Agar (TSA) was spread on an aseptic Petri dish until it was dried. 100 μL of bacterial suspension cultured to logarithmic phase was added to 5 mL of semisolid medium supplemented with 0.4% TSB, mixed well, spread on to the dried agar plate, and naturally dried to solidify the soft agar. 2 μL of phage medium was added on the soft agar by the spotting method, naturally dried and cultured at 37° C. for 12 h. Results were divided into clear plaques (+) in the spotting zone and no plaque (−) in the spotting zone.

Thirteen laboratory-preserved Burkholderia gladioli strains were selected. Of them, Burkholderia gladioli ATCC 33664 was purchased from BeNa Culture Collection, and the remaining 12 strains were Burkholderia gladioli strains isolated and identified from black fungus, golden needle mushroom, and shiitake mushroom purchased from different vegetable markets in Hefei from 2021 to 2022; selected 10 Vibrio parahemolyticus strains were isolated from the food by the Institute of Microbiology, Guangdong Academy of Sciences; and the remaining strains were deposited in Hefei University of Technology.

TABLE 1
Host spectrum of the phage

| No. | Bacterial species | Strain name | Lytic effect |
|---|---|---|---|
| 1 | Burkholderia gladioli | ATCC33664 | ++ |
| 2 | Burkholderia gladioli | BG001 | + |
| 3 | Burkholderia gladioli | BG002 | + |
| 4 | Burkholderia gladioli | BG003 | +++ |
| 5 | Burkholderia gladioli | BG004 | +++ |
| 6 | Burkholderia gladioli | BG006 | +++ |
| 7 | Burkholderia gladioli | BG007 | +++ |
| 8 | Burkholderia gladioli | BG008 | + |
| 9 | Burkholderia gladioli | BG015 | + |
| 10 | Burkholderia gladioli | BG016 | +++ |
| 11 | Burkholderia gladioli | BG018 | + |
| 12 | Burkholderia gladioli | BG019 | + |
| 13 | Burkholderia gladioli | BG024 | +++ |
| 14 | Vibrio parahemolyticus | WT60 | − |
| 15 | Vibrio parahemolyticus | WT64 | − |
| 16 | Vibrio parahemolyticus | WT78 | − |
| 17 | Vibrio parahemolyticus | WT80 | − |
| 18 | Vibrio parahemolyticus | WT81 | − |
| 19 | Vibrio parahemolyticus | WT83 | − |
| 20 | Vibrio parahemolyticus | WT85 | − |
| 21 | Vibrio parahemolyticus | WT89 | − |
| 22 | Vibrio parahemolyticus | WT91 | − |
| 23 | Vibrio parahemolyticus | WT92 | − |
| 24 | Klebsiella pneumoniae | WT01 | − |
| 25 | Klebsiella pneumoniae | WT02 | − |
| 26 | Klebsiella pneumoniae | WT03 | − |
| 27 | Klebsiella pneumoniae | WT04 | − |
| 28 | Klebsiella pneumoniae | WT05 | − |
| 29 | Klebsiella pneumoniae | WT06 | − |
| 30 | Klebsiella pneumoniae | WT07 | − |
| 31 | Klebsiella pneumoniae | WT08 | − |
| 32 | Klebsiella pneumoniae | WT09 | − |
| 33 | Klebsiella pneumoniae | WT10 | − |
| 34 | Cronobacter sakazakii | cro2375w | − |
| 35 | Cronobacter sakazakii | cro3525w | − |
| 36 | Cronobacter sakazakii | cro2451A1 | − |
| 37 | Cronobacter sakazakii | cro2224A2 | − |
| 38 | Cronobacter sakazakii | cro1931w | − |
| 39 | Cronobacter turicensis | cro3005A1 | − |
| 40 | Cronobacter turicensis | cro2864C1 | − |
| 41 | Cronobacter muytjensii | cro1187W | − |
| 42 | Cronobacter muytjensii | cro1187A3 | − |
| 43 | Cronobacter dublinensis | cro981C3 | − |
| 44 | Cronobacter dublinensis | cro2864A2 | − |
| 45 | Cronobacter condimenti | LMG 26250 | − |
| 46 | Salmonella | 72-5 | − |
| 47 | Salmonella | 72-1 | − |
| 48 | Escherichia coli | 3372A1 | − |
| 49 | Escherichia coli | 3466A5 | − |
| 50 | Escherichia coli | 2627-2 | − |
| 51 | Escherichia coli | 10813 | − |
| 52 | Staphylococcus aureus | 295 | − |
| 53 | Staphylococcus aureus | 313 | − |
| 54 | Staphylococcus aureus | 3620 | − |
| 55 | Pseudomonas aeruginosa | PAO1 | − |

It can seen from Table 1 that Burkholderia gladioli phage vB_BglM_WTB has a lytic effect on all of 13 Burkholderia gladioli strains and has no lytic effect on non-Burkholderia gladioli strains.

### Example 4

### Phage Genome Sequencing

After enrichment culture of a single phage strain, phage genomic DNA was extracted by the phenol-chloroform-isopropyl alcohol method. The extracted DNA pellets were dissolved in sterile water and stored at −20° C. for later use. Illumina was used for whole genome sequencing after the concentration and purity of the DNA solution were determined to be up to the standard.

Sequencing results indicated that the genome of Burkholderia gladioli phage vB_BglM_WTB has a full length of 68,541 bp and a GC content of 60.04%.

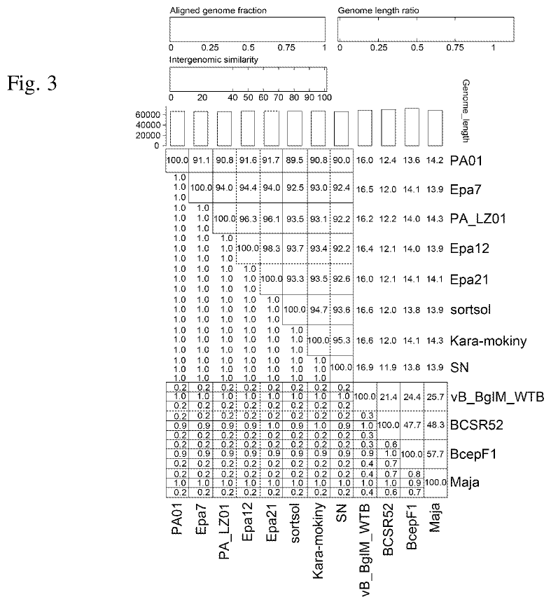

The whole genome sequence of phages with high similarity to Burkholderia gladioli phage vB_BglM_WTB were downloaded from the NCBI database. VIRIDIC online tool was used to plot a heatmap shown in FIG. 3. The results showed that Burkholderia gladioli phage vB_BglM_WTB had low similarity to other phages, and the similarity to Burkholderia phage Maja was the highest, 25.7% (<50%). Visibly, the phage is a new species, and it is recommended that the phage constitutes a new genus.

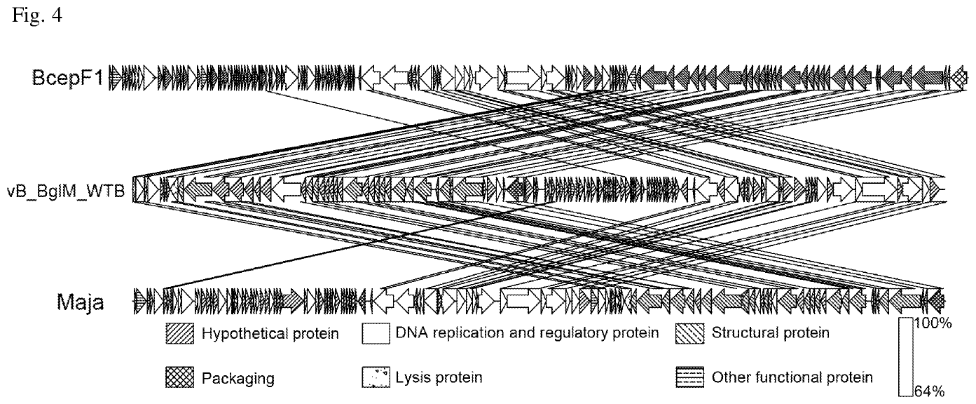

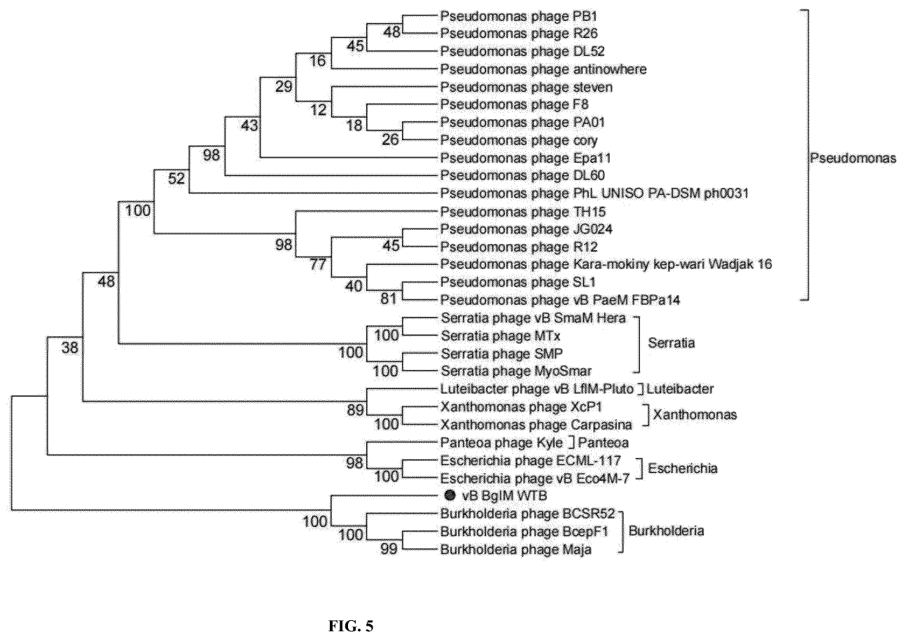

FIG. 4 shows the visual analysis of its proteins. Burkholderia gladioli phage vB_BglM_WTB is expected to have 112 open reading frames (ORFs), 39 of which are annotated as functional proteins and 73 of which are annotated as hypothetical proteins. All functional proteins can be divided into five modules, including DNA metabolic module, lysis module, packaging module, structure module, and other functional modules. FIG. 5 shows its phylogenetic tree of major capsid proteins. The phage is individually located on a branch and has a far genetic relationship with other phages, which may share a common ancestor with Burkholderia gladioli phage.

### Example 5

### Optimal MOI Assay

According to MOIs of 100:1, 10:1, 1:1, 1:10, 1:100, 1:1,000, 1:10,000, 1:100,000, and 1:1,000,000, the phage proliferation liquid and the logarithmic host bacterial suspension were added to TSB, and the total volume of the culture system was guaranteed to be the same. After shake culture at 200 rpm for 8 h at 37° C., the culture system was centrifuged at 12,000 g for 10-15 min at 4° C., and its titer was assayed by the double-layer agar plate method. The results are shown in Table 2.

TABLE 2
MOI

| MOI | pfu/mL |
|---|---|
| 100:1 | 2.50 × 10⁷ |
| 10:1 | 9.40 × 10⁷ |
| 1:1 | 1.01 × 10⁸ |
| 1:10 | 1.81 × 10⁸ |
| 1:100 | 9.30 × 10⁸ |
| 1:1000 | 1.07 × 10⁹ |
| 1:10000 | 1.15 × 10⁹ |
| 1:100000 | 4.70 × 10⁸ |
| 1:1000000 | 2.60 × 10⁸ |

The results show that the optimal MOI of Burkholderia gladioli phage vB_BglM_WTB is 1:10,000.

### Example 6

### Determination of One-Step Growth Curve

Burkholderia gladioli ATCC 33664 was cultured to the pre-logarithmic phase to allow the bacterial concentration to be 10⁸ cfu/mL; the host and the phage medium were added according to the optimal MOI of 1:10,000; after water bath at 37° C. for 5 min, the culture system was centrifuged at 12,000 rpm for 30 s, and the supernatant was discarded. The pellets were washed with TSB twice. 30 mL of TSB preheated at 37° C. was added for shake culture at 37° C. Sampling was performed at 0 min and every 10 min within the first 60 min, followed by every 30 min. The sample was centrifuged at 12,000 rpm for 30 s and filtrated through a 0.45 μm filter head. The phage titer was assayed at each time point. A one-step growth curve was plotted with infection time as abscissa and phage titer as ordinate.

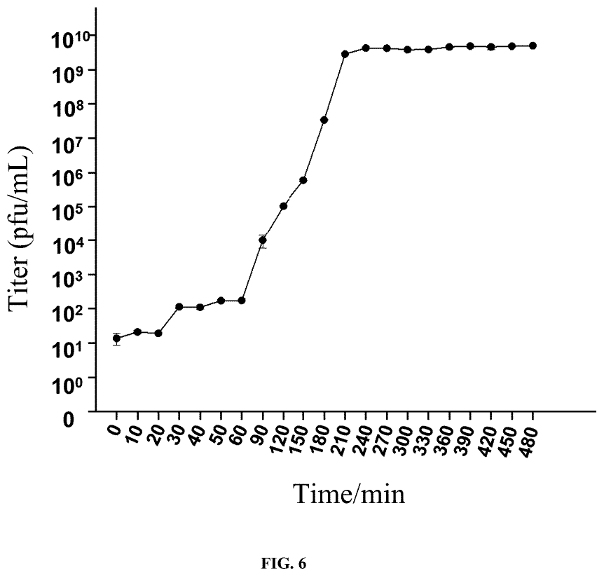

The result is shown in FIG. 6, where 0-60 min is a latent period of the phage, 60-210 min is a lysis period of the phage, and 210-480 min is a plateau of the phage.

### Example 7

### Determination of pH Stability

The pH values of TSB were adjusted with dilute hydrochloric acid and dilute sodium hydroxide solution, and TSBs at pH 2, 3, 4, 5, 6, 7, 8, 9, 10, 11, and 12 were prepared. TSBs at different pH values were filtered through a 0.45 μm millipore filter to remove bacteria, the phage was diluted to 10⁹ pfu/mL with TSB, diluents were held in water bath at 37° C. for 1 h, and the titer was assayed by the double-layer agar plate method.

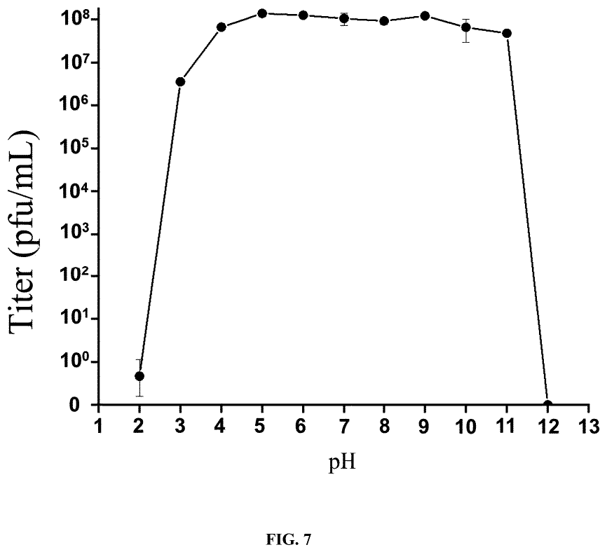

The results are shown in FIG. 7. Burkholderia gladioli phage vB_BglM_WTB is stable at pH 3-11, with a titer of around 10⁸ pfu/mL; when the pH value is higher than 11, the phage titer begins dropping; when the pH value reaches 12, the phage could hardly survive. The results show that the optimum growth pH of Burkholderia gladioli phage vB_BglM_WTB is neutral-to-alkaline.

### Example 8

### Determination of Thermal Stability

The phage stock solution (10⁹ pfu/mL) was dispensed into EP tubes, and incubated at 25, 30, 37, 40, 45, 50, 55, 60, 65, 70, 75, and 80° C. for 60 min, respectively; after the phage stock solution was diluted appropriately, the titer was assayed by the double-layer agar plate method.

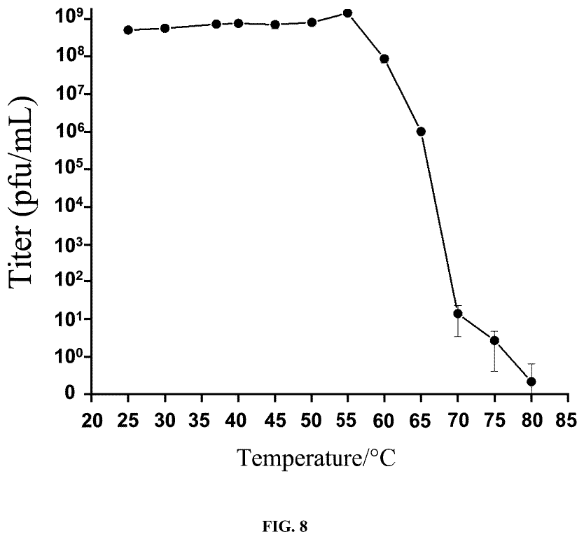

The results are shown in FIG. 8. As the temperature rises, the phage activity becomes lower and lower. After action at 65° C. for 60 min, the titer can still reach 1.01×10⁶ pfu/mL, indicating that the phage has certain temperature tolerance; after action at 80° C. for 60 min, the phage could hardly survive.

### Example 9

### Bactericidal Effect of the Phage on Black Fungus

Some dried black fungi were soaked in sterile water for 10 min; 0.5(±0.05) g of equal-sized wet black fungi with a size of 2×2 cm were weighed; equal-sized black fungi were put in 90° C. sterile water for water bath for 30 min and transferred into a clean bench for ultraviolet irradiation for 2 h. Each side of a black fungus was irradiated for 1 h. The treated black fungi were soaked in a Burkholderia gladioli BG007 suspension (10⁸ cfu/mL) for 10 min; the black fungi were air-dried and transferred into a phage stock solution (10⁹ pfu/mL) for soaking for 10 min; the black fungi were air-dried at room temperature, transferred onto a clean and aseptic Petri dish, and sampled at 0, 2, 4, 6, 8, 10, and 12 h; the black fungi were cut into pieces, re-suspended in phosphate buffered saline (PBS), and serially diluted to determine the bacterial count.

The calculation formula of the sterilization rate is: (Burkholderia gladioli count of the control group−Burkholderia gladioli count of the experimental group)/Burkholderia gladioli count of the control group×100%.

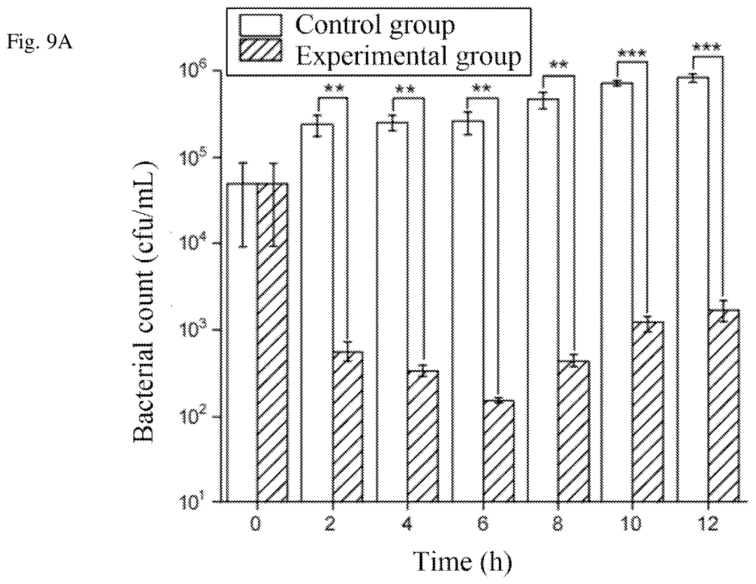

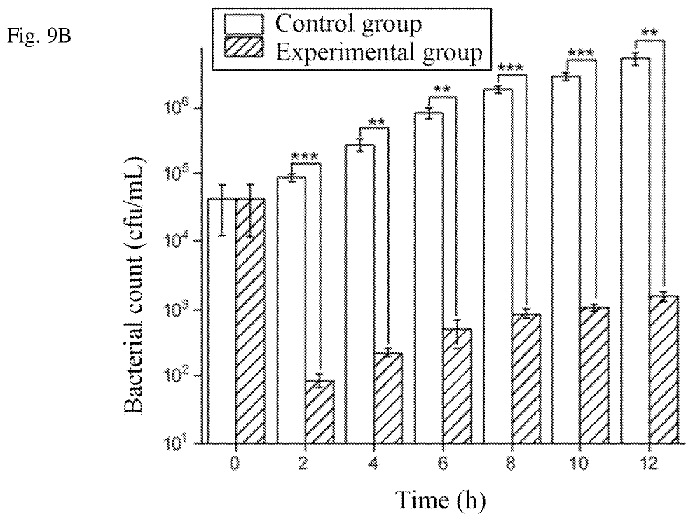

The results are shown in FIGS. 9A-B. From FIG. 9A, at 4° C., the bacterial count of the phage-treated black fungi is minimized after 6 h, namely 154 cfu/mL, which is reduced by 2.6×10⁵ cfu/mL (5.42 log) compared with that of the control group, during which the sterilization rate of Burkholderia gladioli phage vB_BglM_WTB in black fungi reaches 99.94%; after treatment for 12 h, the bacterial count of the phage-treated black fungi rises slightly, during which the bacterial count reaches 1,710 cfu/mL, but can still be reduced by 8.38×10⁵ cfu/mL (5.92 log) compared with that of the control group. From FIG. 9B, at 25° C., the bacterial count of the phage-treated black fungi is minimized after 2 h, only 89 cfu/mL, which is reduced by 8.5×10⁴ cfu/mL (4.93 log), with a sterilization rate of 99.90%; after treatment for 12 h, the bacterial count rises slightly to 1,546 cfu/mL, but can still be reduced by 5.51×10⁶ cfu/mL (6.71 log) compared with that of the control group.

From FIGS. 9A and 9B, compared with results of two temperatures, treatment at 25° C. obtains a better effect, the bacterial count can be minimized at 2 h after treatment, and fewer bacteria survive. It is indicated that the host killing effect is excellent at 25° C. with short action time; due to low survival rate of the strain and short survival time, the possibility of toxin production is reduced and the edible safety of food like black fungus is enhanced.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

1. A phage, wherein the phage is Burkholderia gladioli phage vB_BglM_WTB with an accession number of CCTCC NO: M 2023525.

2. The phage according to claim 1, wherein a genome of the phage has a full length of 68,541 bp and contains no virulence gene or drug resistance gene.

3. The phage according to claim 1, wherein the phage is stably active at pH 3-11 and has a tolerable temperature of 25-65° C.

4. A lysate, wherein the lysate is a lysate of the phage according to claim 1.

5. A preparation, comprising the phage according to claim 1.

6-20. (canceled)

21. The lysate according to claim 4, wherein a genome of the phage has a full length of 68,541 bp and contains no virulence gene or drug resistance gene.

22. The lysate according to claim 4, wherein the phage is stably active at pH 3-11 and has a tolerable temperature of 25-65° C.

23. The preparation according to claim 5, wherein a genome of the phage has a full length of 68,541 bp and contains no virulence gene or drug resistance gene.

24. The preparation according to claim 5, wherein the phage is stably active at pH 3-11 and has a tolerable temperature of 25-65° C.

25. A preparation, comprising the lysate according to claim 4.

26. The preparation according to claim 25, wherein a genome of the phage has a full length of 68,541 bp and contains no virulence gene or drug resistance gene.

27. The preparation according to claim 25, wherein the phage is stably active at pH 3-11 and has a tolerable temperature of 25-65° C.

28. A method for controlling Burkholderia gladioli using the phage according to claim 1.

29. The method according to claim 28, wherein a genome of the phage has a full length of 68,541 bp and contains no virulence gene or drug resistance gene.

30. The method according to claim 28, wherein the phage is stably active at pH 3-11 and has a tolerable temperature of 25-65° C.

31. A method for controlling Burkholderia gladioli using the lysate according to claim 4.

32. The method according to claim 31, wherein the method is intended to control the Burkholderia gladioli in food.

33. The method according to claim 31, wherein the phage has a multiplicity of infection (MOI) of 1:10,000-1:100.

34. The method according to claim 31, wherein the phage, the lysate or the preparation is in contact with the Burkholderia gladioli.

35. The method according to claim 34, wherein a pH value of a contact medium is 3-11, and contact time is at least 2 h.
